(19) United States
(12) Patent Application Publication
(10) Pub. No.: US 2022/0015478 A1
(43) Pub. Date: Jan. 20, 2022

(54) DETACHABLE BI-LAYERED GLOVE AND METHOD OF MANUFACTURING THEREOF

(71) Applicant: Top Glove International Sdn. Bhd., Klang (MY)

(72) Inventors: Chong Ban WONG, Klang (MY); Siew Szen LING, Klang (MY); Meng Lai LOW, Klang (MY); Sonia How Ming WONG, Klang (MY)

(21) Appl. No.: 17/371,719

(22) Filed: Jul. 9, 2021

(30) Foreign Application Priority Data
Jul. 15, 2020 (MY) PI2020003651

(51) Int. Cl. A41D 19/00; A61B 42/10; B29C 41/14; B29C 41/22

(52) U.S. Cl. CPC A41D 19/0082; A41D 19/001; A61B 42/10; B29L 2031/4864; B29C 41/22; A41D 2500/54; A41D 2400/70; B29C 41/14

(57) ABSTRACT

A detachable glove comprising at least two layers that are separated by an adhesive layer, wherein the adhesive layer includes a wetting agent, a latex coagulating agent, a synthetic organic polymer and water. A chlorinated detachable bi-layered glove and a method of manufacturing thereof. A polymer coated, detachable, bi-layered glove and a method of manufacturing thereof. A method of peeling a detachable bi-layered glove wherein the method includes (i) pulling cuff area of the outer glove layer until its circumference is detached from the inner glove layer and (ii) peeling off the outer glove layer once the full circumference of the outer glove layer is detached from the inner glove layer to yield the inner glove layer for further usage.

### CROSS-REFERENCE TO RELATED APPLICATION

This application claims priority under 35 U.S.C. § 119 to Malaysia Patent Application No. PI2020003651, filed Jul. 15, 2020, the disclosure of which is incorporated by reference herein in its entirety.

### FIELD OF THE INVENTION

The present invention relates to a detachable bi-layered glove and method of manufacturing thereof, which provide users the convenience of using the same piece of the glove twice without the risk of contamination and the hassle of doffing and donning twice.

### BACKGROUND OF THE INVENTION

In numerous industries such as healthcare, pharmaceutical and food, double gloving has been a common practice. Simply, double layered protection has become a preferable option when users deal with high risk chemicals. Double gloving instead of single gloving is advisable since it provides extra protection and it reduces the risk of contamination. Further, single gloving has a major drawback, whereby the users tend to doff the used single-layered glove and don a fresh piece of the glove over sweaty hands, which is actually taxing as well as causes discomfort.

Meanwhile, implementation of double gloving in the healthcare field was influenced by various factors such as but not limited to surgical procedure, risk status of patient, abrasion on the hands of the surgical team and personal preference. Similarly, cross-contamination between human hands and food products was the main reason for the implementation of double gloving in the food industry.

However, one major setback faced by users in double gloving is that the users require additional time for donning the second glove over the first glove which will directly affect the efficiency of the users. Thus, from the above, it is obvious that the glove manufacturing industries are facing some challenges to overcome, for which an approach is developed to identify a detachable bi-layered glove and an appropriate method for users to conveniently use the same piece of glove for twice without the risk of contamination and the hassle of doffing and donning for twice.

### SUMMARY OF THE INVENTION

The present invention relates to a detachable glove comprising at least two layers that are separated by an adhesive layer, wherein the adhesive layer includes a wetting agent, a latex coagulating agent, a synthetic organic polymer and water. The present invention also relates to a chlorinated detachable bi-layered glove and method of manufacturing thereof. The present invention further relates to a polymer coated detachable bi-layered glove and method of manufacturing thereof. Still further, the present invention relates to a method of peeling a detachable bi-layered glove wherein the method includes (i) pulling a cuff area of an outer glove layer until its circumference is detached from an inner glove layer and (ii) peeling off the outer glove layer once the full circumference of the outer glove layer is detached from the inner glove layer to yield the inner glove layer for further usage.

Additional aspects, features and advantages of the invention will become apparent to those skilled in the art upon consideration of the following detailed description of preferred embodiments of the invention.

### BRIEF DESCRIPTION OF THE ACCOMPANYING DRAWINGS

The present invention will be fully understood from the detailed description given herein below and the accompanying drawings which are given by way of illustration only, and thus are not limitative of the present invention, wherein:

In the appended drawings:

FIGS. 1a and 1b collectively is a flowchart showing the steps involved in preparing a chlorinated detachable bi-layered glove of the present invention.

FIGS. 2a and 2b collectively is a flowchart showing the steps involved in preparing a polymer coated detachable bi-layered glove of the present invention.

FIG. 3a is an illustrative figure of a glove with a shorter inner glove layer and a longer outer glove layer of the detachable bi-layered glove of the present invention.

FIG. 3b is an illustrative figure showing the method of detaching the longer outer glove layer from the shorter inner glove layer of the detachable bi-layered glove of the present invention.

FIG. 3c is an illustrative figure showing the method of peeling off the longer outer glove layer from the shorter inner glove layer of the detachable bi-layered glove of the present invention.

FIG. 3d is an illustrative figure showing the shorter inner glove layer of the bi-layered glove of the present invention.

FIG. 4a is an illustrative figure of a glove with a longer inner glove layer and a shorter outer glove layer of the detachable bi-layered glove of the present invention.

FIG. 4b is an illustrative figure showing the method of detaching the shorter outer glove layer from the longer inner glove layer of the detachable bi-layered glove of the present invention.

FIG. 4c is an illustrative figure showing the method of peeling off the shorter outer glove layer from the longer inner glove layer of the detachable bi-layered glove of the present invention.

FIG. 4d is an illustrative figure showing the longer inner glove layer of the bi-layered glove of the present invention.

### DETAILED DESCRIPTION OF THE INVENTION

A detailed description of preferred embodiments of the present invention is disclosed herein. It should be understood, however, that the embodiments are merely exemplary of the present invention, which may be embodied in various forms. Therefore, the details disclosed herein are not to be interpreted as limiting, but merely as the basis for the claims and for teaching one skilled in the art of the invention. The numerical data or ranges used in the specification are not to be construed as limiting.

The present invention relates to a detachable bi-layered glove and method of manufacturing thereof, in particular the present invention relates to a detachable bi-layered glove, including, but not limited to, any glove selected from the group consisting of acrylonitrile butadiene rubber (NBR) glove, natural rubber (NR) glove, polychloroprene (CR) rubber glove, polyisoprene (PI) rubber glove, styrene butadiene rubber (SBR) glove, butadiene copolymers rubber glove, polyurethane (PU) glove, polyvinyl chloride (PVC) glove, thermoplastic elastomer (TPE) glove, vinyl acetate ethylene (VAE) glove and mixtures thereof and method of manufacturing thereof which provide users the convenience of using a same piece of the glove twice without the risk of contamination and the hassle of doffing and donning twice.

For the purpose of this invention, the term “bi-layered” can also be construed as two-layered or double-layered which is made of an outer glove layer and an inner glove layer. Simply, the bi-layered glove comprises an outer glove layer and an inner glove layer. For the purpose of this invention, the term “detachable” can also be construed as removable and/or peel off. Further, the term “detachable” is associated to the action of peeling off the outer glove layer from the inner glove layer.

A first embodiment of the present invention relates to a detachable bi-layered glove. The detachable bi-layered glove comprises an outer glove layer and an inner glove layer that is separated by an adhesive layer. The glove may be any glove selected from the group consisting of acrylonitrile butadiene rubber (NBR) glove, natural rubber (NR) glove and polychloroprene (CR) rubber glove, polyisoprene (PI) rubber glove, styrene butadiene rubber (SBR) glove, butadiene copolymers rubber glove, polyurethane (PU) glove, polyvinyl chloride (PVC) glove, thermoplastic elastomer (TPE) glove, vinyl acetate ethylene (VAE) glove and mixtures thereof.

Alternatively, the present invention may also be applied on a detachable multi-layered glove. In an embodiment of the present invention, the multi-layered glove includes at least 3 layers. In an embodiment, each of the layers is separated by an adhesive layer.

The latex to manufacture the glove may be obtained from any commercially available latex suppliers. The latexes are used as 100 phr (also referred to as parts per hundred rubber), wherein the parts per hundred rubber is used as a basis for amounting other components.

The adhesive layer includes at least:
a wetting agent, wherein the wetting agent is used in an amount ranging between 0.40% to 0.80%, and, in some embodiments, preferably 0.60% to 0.70% by weight of the adhesive layer, wherein the wetting agent is selected from the group consisting of non-ionic surfactant, blends of non-ionic surfactant and mixtures thereof. The non-ionic surfactant is selected from the group consisting of fatty acid ethoxylate, lauric acid ethoxylate, oleic acid ethoxylate, other similar compounds and mixtures thereof. The blends of non-ionic surfactant are polysiloxane surfactant which is selected from the group consisting of methyl (propylhydroxide ethoxylated) bis (trimethylsiloxy) silane, other similar compounds and mixtures thereof. In some embodiments, the wetting agent is preferably fatty acid ethoxylate. The wetting agent serves to improve wettability of the adhesive layer;
a latex coagulating agent, wherein the latex coagulating agent is used in an amount ranging between 17.00% to 20.00%, and, in some embodiments, preferably 17.50% to 19.50% by weight of the adhesive layer. The latex coagulating agent is selected from the group consisting of calcium nitrate, zinc nitrate, calcium chloride and mixtures thereof, and in some embodiments, preferably calcium nitrate. The latex coagulating agent serves to coagulate latex dispersion;
a synthetic organic polymer, wherein the synthetic organic polymer is used in an amount ranging between 10.00% to 14.00%, and in some embodiments, preferably 12.00% to 13.00% by weight of the adhesive layer. The synthetic organic polymer is selected from the group consisting of styrene acrylic copolymer emulsion, polymethyl methacrylate emulsion, low-density polyethylene emulsion, high-density polyethylene emulsion, polypropylene emulsion, polystyrene emulsion, polyurethane emulsion and mixtures thereof, and in some embodiments, preferably styrene acrylic copolymer emulsion or polymethyl methacrylate emulsion, and in some embodiments, preferably styrene acrylic copolymer emulsion. The synthetic organic polymer serves to increase block resistance for efficient peeling off the outer glove layer from the inner glove layer; and
a water, wherein the water is used in an amount ranging between 65.20% to 72.60%, and in some embodiments, preferably 66.80% to 69.90% by weight of the adhesive layer. The water serves as a diluent.

Table 1 shows chemical components and compositions thereof (as mentioned above) used in preparing the adhesive layer.

TABLE 1
Chemical components and compositions
thereof to prepare the adhesive layer

| | Working Range | Preferred range |
|---|---|---|
| Chemicals | (Weight %) | (Weight %) |
| Wetting agent | 0.40% to 0.80% | 0.60% to 0.70% |
| Latex coagulating agent | 17.00% to 20.00% | 17.50% to 19.50% |
| Synthetic organic polymer | 10.00% to 14.00% | 12.00% to 13.00% |
| Diluent | 65.20% to 72.60% | 66.80% to 69.90% |

The outer glove layer and the inner glove layer are in different lengths for easy removal. Simply, if the outer glove layer is longer, then the inner glove layer would be shorter and vice versa. The longer glove layer has a standard length ranging between 270 mm to 290 mm, whereas the shorter glove layer has a standard length ranging between 230 mm to 250 mm.

A second embodiment of the present invention relates to a method to prepare the detachable bi-layered glove (as stated above). The detachable bi-layered glove may be a chlorinated and/or polymer coated detachable bi-layered glove.

### Chlorinated Detachable Bi-Layered Glove

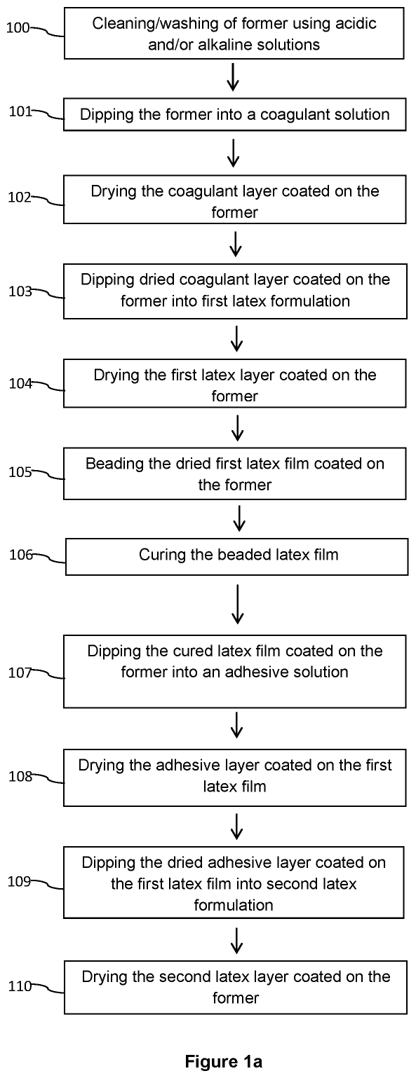

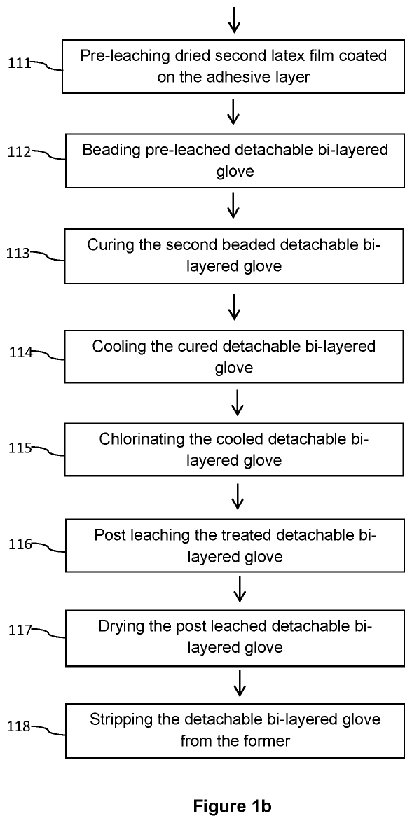

Referring to the accompanying drawing, FIGS. 1a and 1b collectively is a flowchart showing a method of preparing a chlorinated detachable bi-layered glove, wherein the method comprises the steps of:
i. cleaning/washing former firstly with acidic solution at a temperature ranging between 50° C. to 70° C. for a duration ranging between 3 seconds to 10 seconds, and in some embodiments, preferably between 5 seconds to 7 seconds, secondly (optionally) water rinsing at a temperature ranging between 50° C. to 70° C. for a duration ranging between 3 seconds to 10 seconds, and in some embodiments, preferably between 5 seconds to 7 seconds, followed by cleaning/washing with alkaline solution at a temperature ranging between 50° C. to 70° C. for a duration ranging between 5 seconds to 15 seconds, and in some embodiments, preferably between 10 seconds to 12 seconds and lastly water rinsing at a temperature ranging between 55° C. to 75° C. for a duration ranging between 5 seconds to 15 seconds, and in some embodiments, preferably between 10 seconds to 12 seconds to produce cleaned former (100);
ii. dipping the former obtained in step (i) into a coagulant solution to produce a coagulant layer coated on the former, wherein the dipping is carried out at a temperature ranging between 50° C. to 70° C., and in some embodiments, preferably between 60° C. to 65° C. for a duration ranging between 8 seconds to 12 seconds, and in some embodiments, preferably between 9 seconds to 10 seconds, wherein the coagulant solution is any conventional coagulant solution (101);
iii. drying the coagulant layer coated on the former obtained in step (ii) to produce a dried coagulant layer coated on the former, wherein the drying is carried out at a temperature ranging between 100° C. to 180° C., and in some embodiments, preferably between 130° C. to 150° C. for a duration ranging between 50 seconds to 180 seconds, and in some embodiments, preferably between 60 seconds to 120 seconds (102);
iv. dipping the dried coagulant layer coated on the former obtained in step (iii) into first latex formulation to produce a first latex layer coated on the former (which is the outer glove layer of the detachable bi-layered glove), wherein the dipping is carried out at a temperature ranging between 50° C. to 70° C., and in some embodiments, preferably between 60° C. to 65° C. for a duration ranging between 8 seconds to 12 seconds, and in some embodiments, preferably between 9 seconds to 10 seconds (103);
v. drying the first latex layer coated on the former obtained in step (iv) to produce a dried first latex film coated on the former, wherein the drying is carried out at a temperature ranging between 100° C. to 150° C., and in some embodiments, preferably between 120° C. to 140° C. for a duration ranging between 50 seconds to 180 seconds, and in some embodiments, preferably between 60 seconds to 120 seconds (104);
vi. beading the dried first latex film coated on the former obtained in step (v) to produce a beaded latex film coated on the former, wherein the beading is performed using any conventional beading carpet or beading brush found in the production line (105);
vii. curing the beaded latex film coated on the former obtained in step (vi) to produce a cured latex film coated on the former, wherein the curing is carried out at a temperature ranging between 100° C. to 150° C., and in some embodiments, preferably between 120° C. to 140° C. for a duration ranging between 8 minutes to 20 minutes, and in some embodiments, preferably between 10 minutes to 15 minutes (106);
viii. dipping the cured latex film coated on the former obtained in step (vii) into an adhesive solution to produce an adhesive layer coated on the first latex film, wherein the dipping is carried out at a temperature ranging between 50° C. to 70° C., and in some embodiments, preferably between 60° C. to 65° C. for a duration ranging between 8 seconds to 12 seconds, and in some embodiments, preferably between 9 seconds to 10 seconds (107);
ix. drying the adhesive layer coated on the first latex film obtained in step (viii) to produce a dried adhesive layer coated on the first latex film, wherein the drying is carried out at a temperature ranging between 100° C. to 180° C., and in some embodiments, preferably between 130° C. to 150° C. for a duration ranging between 50 seconds to 180 seconds, and in some embodiments, preferably between 60 seconds to 120 seconds (108);
x. dipping the dried adhesive layer coated on the first latex film obtained in step (ix) into second latex formulation to produce a second latex layer coated on the adhesive layer (which is the inner glove layer of the detachable bi-layered glove), wherein the dipping is carried out at a temperature ranging between 50° C. to 70° C., and in some embodiments, preferably between 60° C. to 65° C. for a duration ranging between 8 seconds to 12 seconds, and in some embodiments, preferably between 9 seconds to 10 seconds (109);
xi. drying the second latex layer coated on the adhesive layer obtained in step (x) to produce a dried second latex film coated on the adhesive layer wherein the drying is carried out at a temperature ranging between 100° C. to 150° C., and in some embodiments, preferably between 120° C. to 140° C. for a duration ranging between 50 seconds to 180 seconds, and in some embodiments, preferably between 60 seconds to 120 seconds (110);
xii. pre-leaching the dried second latex film coated on the adhesive layer obtained in step (xi) to produce a pre-leached detachable bi-layered latex film coated on the former, wherein the pre-leaching is carried out at a temperature ranging between 50° C. to 70° C., and in some embodiments, preferably between 55° C. to 60° C. for a duration ranging between 15 seconds to 40 seconds, and in some embodiments, preferably between 20 seconds to 30 seconds (111);
xiii. beading the pre-leached detachable bi-layered latex film coated on the former obtained in step (xii) wherein the beading is performed using any conventional beading carpet or beading brush found in the production line to produce a beaded detachable bi-layered latex film on the surface (112);
xiv. curing the beaded detachable bi-layered latex film coated on the former obtained in step (xiv) to produce a cured detachable bi-layered glove coated on the former, wherein the curing is carried out at a temperature ranging between 110° C. to 150° C., and in some embodiments, preferably between 120° C. to 140° C. for a duration ranging between 8 minutes to 20 minutes, and in some embodiments, preferably between 10 minutes to 15 minutes (113);
xv. cooling the cured detachable bi-layered glove coated on the former obtained in step (xiv) to produce a cooled detachable bi-layered glove coated on the former, wherein the cooling is carried out to bring down the temperature to a temperature ranging between 23° C. to 30° C., and in some embodiments, preferably between 25° C. to 27° C. for a duration ranging between 8 seconds to 20 seconds, and in some embodiments, preferably between 10 seconds to 15 seconds (114);
xvi. chlorinating the cooled detachable bi-layered glove coated on the former obtained in step (xv) to produce a chlorine treated detachable bi-layered glove coated on the former, wherein the chlorination is carried out at a temperature ranging between 23° C. to 30° C., and in some embodiments, preferably between 25° C. to 27° C. for a duration ranging between 8 seconds to 20 seconds, and in some embodiments, preferably between 10 seconds to 15 seconds (115);
xvii. post-leaching the chlorine treated detachable bi-layered glove coated on the former obtained in step (xvi) to produce a post-leached detachable bi-layered glove coated on the former, wherein the post-leaching is carried out at a temperature ranging between 50° C. to 70° C., and in some embodiments, preferably between 55° C. to 60° C. for a duration ranging between 15 seconds to 40 seconds, and in some embodiments, preferably between 20 seconds to 30 seconds (116);
xviii. drying the post-leached detachable bi-layered glove coated on the former obtained in step (xvii) to produce the chlorinated detachable bi-layered glove, wherein the drying is carried out at a temperature ranging between 100° C. to 180° C., and in some embodiments, preferably 130° C. to 150° C. for a duration ranging between 50 seconds to 180 seconds, and in some embodiments, preferably between 60 seconds to 120 seconds (117);
xix. stripping the chlorinated detachable bi-layered glove from the former (118), and
wherein the first and second latex formulations and resultant gloves thereof are of identical formulations, details of which is described below, wherein the first and the second gloves are in different lengths, wherein the longer glove has a standard length ranging between 270 mm to 290 mm, wherein the shorter glove has a standard length ranging between 230 mm to 250 mm.

### Polymer Coated Detachable Bi-Layered Glove

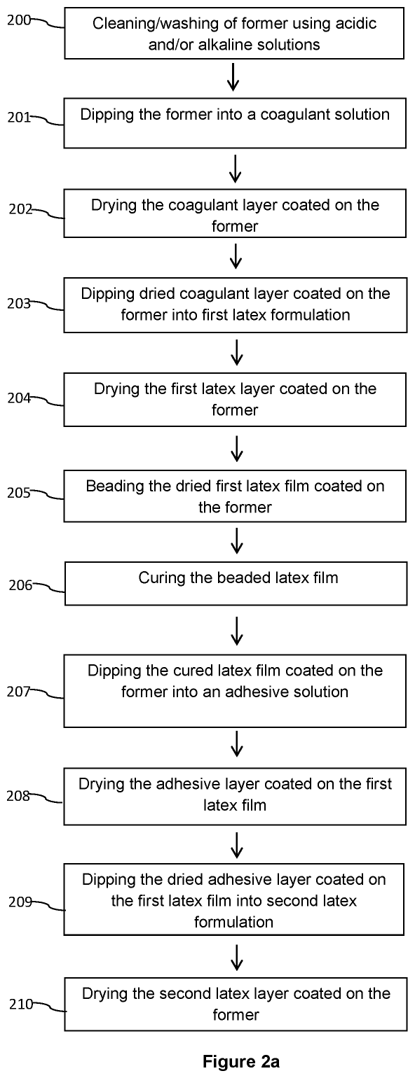

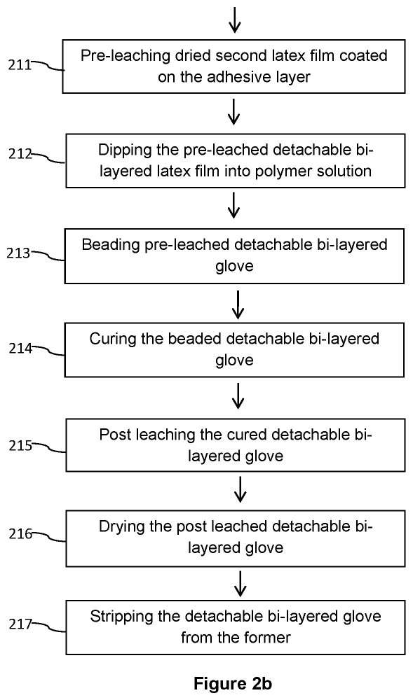

Referring to the accompanying drawing, FIGS. 2a and 2b collectively is a flowchart showing a method of preparing a polymer coated detachable bi-layered glove, wherein the method comprises the steps of:
i. cleaning/washing former firstly with acidic solution at a temperature ranging between 50° C. to 70° C. for a duration ranging between 3 seconds to 10 seconds, and in some embodiments, preferably between 5 seconds to 7 seconds, secondly (optionally) water rinsing at a temperature ranging between 50° C. to 70° C. for a duration ranging between 3 seconds to 10 seconds, and in some embodiments, preferably between 5 seconds to 7 seconds, followed by cleaning/washing with alkaline solution at a temperature ranging between 50° C. to 70° C. for a duration ranging between 5 seconds to 15 seconds, and in some embodiments, preferably between 10 seconds to 12 seconds and lastly water rinsing at a temperature ranging between 55° C. to 75° C. for a duration ranging between 5 seconds to 15 seconds, and in some embodiments, preferably between 10 seconds to 12 seconds to produce cleaned former (200);
ii. dipping the cleaned former obtained in step (i) into a coagulant solution to produce a coagulant layer coated on the former, wherein the dipping is carried out at a temperature ranging between 50° C. to 70° C., and in some embodiments, preferably between 60° C. to 65° C. for a duration ranging between 8 seconds to 12 seconds, and in some embodiments, preferably between 9 seconds to 10 seconds, wherein the coagulant solution is any conventional coagulant solution (201);
iii. drying the coagulant layer coated on the former obtained in step (ii) to produce a dried coagulant layer coated on the former, wherein the drying is carried out at a temperature ranging between 100° C. to 180° C., and in some embodiments, preferably between 130° C. to 150° C. for a duration ranging between 50 seconds to 180 seconds, and in some embodiments, preferably between 60 seconds to 120 seconds (202);
iv. dipping the dried coagulant layer coated on the former obtained in step (iii) into first latex formulation to produce a first latex layer coated on the former (which is the outer glove layer of the detachable bi-layered glove), wherein the dipping is carried out at a temperature ranging between 50° C. to 70° C., and in some embodiments, preferably between 60° C. to 65° C. for a duration ranging between 8 seconds to 12 seconds, and in some embodiments, preferably between 9 seconds to 10 seconds (203);
v. drying the first latex layer coated on the former obtained in step (iv) to produce a dried first latex film coated on the former, wherein the drying is carried out at a temperature ranging between 100° C. to 150° C., and in some embodiments, preferably between 120° C. to 140° C. for a duration ranging between 50 seconds to 180 seconds, and in some embodiments, preferably between 60 seconds to 120 seconds (204);
vi. beading the dried first latex film coated on the former obtained in step (v) to produce a beaded latex film coated on the former, wherein the beading is performed using any conventional beading carpet or beading brush found in the production line (205);
vii. curing the beaded latex film coated on the former obtained in step (vi) to produce a cured latex film coated on the former, wherein the curing is carried out at a temperature ranging between 100° C. to 150° C., and in some embodiments, preferably between 120° C. to 140° C. for a duration ranging between 8 minutes to 20 minutes, and in some embodiments, preferably between 10 minutes to 15 minutes (206);
viii. dipping the cured latex film coated on the former obtained in step (vii) into an adhesive solution to produce an adhesive layer coated on the first latex film, wherein the dipping is carried out at a temperature ranging between 50° C. to 70° C., and in some embodiments, preferably between 60° C. to 65° C. for a duration ranging between 8 seconds to 12 seconds, and in some embodiments, preferably between 9 seconds to 10 seconds (207);
ix. drying the adhesive layer coated on the first latex film obtained in step (viii) to produce a dried adhesive layer coated on the first latex film, wherein the drying is carried out at a temperature ranging between 100° C. to 180° C., and in some embodiments, preferably between 130° C. to 150° C. for a duration ranging between 50 seconds to 180 seconds, and in some embodiments, preferably between 60 seconds to 120 seconds (208);
x. dipping the dried adhesive layer coated on the first latex film obtained in step (ix) into second latex formulation to produce a second latex layer coated on the adhesive layer (which is the inner glove layer of the detachable bi-layered glove), wherein the dipping is carried out at a temperature ranging between 50° C. to 70° C., and in some embodiments, preferably between 60° C. to 65° C. for a duration ranging between 8 seconds to 12 seconds, and in some embodiments, preferably between 9 seconds to 10 seconds (209);
xi. drying the second latex layer coated on the adhesive layer obtained in step (x) to produce a dried second latex film coated on the adhesive layer wherein the drying is carried out at a temperature ranging between 100° C. to 150° C., and in some embodiments, preferably between 120° C. to 140° C. for a duration ranging between 50 seconds to 180 seconds, and in some embodiments, preferably between 60 seconds to 120 seconds (210);
xii. pre-leaching the dried second latex film coated on the adhesive layer obtained in step (xi) to produce a pre-leached detachable bi-layered latex film coated on the former, wherein the pre-leaching is carried out at a temperature ranging between 50° C. to 70° C., and in some embodiments, preferably between 55° C. to 60° C. for a duration ranging between 15 seconds to 40 seconds, and in some embodiments, preferably between 20 seconds to 30 seconds (211);
xiii. dipping the pre-leached detachable bi-layered latex film obtained in step (xii) into polymer solution to produce a polymer coated detachable bi-layered latex film, wherein the dipping is carried out at a temperature ranging between 25° C. to 45° C., and in some embodiments, preferably between 35° C. to 40° C. for a duration ranging between 8 seconds to 15 seconds, and in some embodiments, preferably between 10 seconds to 12 seconds (212);
xiv. beading the polymer coated detachable bi-layered latex film coated on the former obtained in step (xiii), wherein the beading is performed using any conventional beading carpet or beading brush found in the production line to produce beaded a detachable bi-layered latex film on the surface (213);
xv. curing the beaded detachable bi-layered latex film coated on the former obtained in step (xiv) to produce a cured detachable bi-layered glove coated on the former, wherein the curing is carried out at a temperature ranging between 110° C. to 150° C., and in some embodiments, preferably between 120° C. to 140° C. for a duration ranging between 8 minutes to 20 minutes, and in some embodiments, preferably between 10 minutes to 15 minutes (214);
xvi. post-leaching the cured detachable bi-layered glove coated on the former obtained in step (xv) to produce post-leached detachable bi-layered glove coated on the former, wherein the post-leaching is carried out at a temperature ranging between 50° C. to 70° C., and in some embodiments, preferably between 55° C. to 60° C. for a duration ranging between 15 seconds to 40 seconds, and in some embodiments, preferably between 20 seconds to 30 seconds (215);
xvii. drying the post-leached detachable bi-layered glove coated on the former obtained in step (xvi) to produce the polymer coated detachable bi-layered glove, wherein the drying is carried out at a temperature ranging between 100° C. to 180° C., and in some embodiments, preferably 130° C. to 150° C. for a duration ranging between 50 seconds to 180 seconds, and in some embodiments, preferably between 60 seconds to 120 seconds (216);
xviii. stripping the polymer coated detachable bi-layered glove from the former (217), and
wherein the first and second latex formulations and resultant gloves thereof are of identical formulations, details of which is described below, wherein the first and the second gloves are in different lengths, wherein the longer glove has a standard length ranging between 270 mm to 290 mm, wherein the shorter glove has a standard length ranging between 230 mm to 250 mm.

The coagulant layer includes:
wetting agent (such as but not limited to fatty acid ethoxylate);
former release agent (such as but not limited to potassium stearate, calcium stearate and calcium carbonate);
latex coagulating agent (such as but not limited to calcium nitrate);
pH adjuster (such as but not limited to ammonia); and
diluent (such as but not limited to water).

Tables 3, 5 and 7 show chemical components (as listed above) and compositions thereof used in preparing the coagulant layer that is in accordance to the latex formulations to be prepared.

The first and the second latex formulations include:
latex (such as but not limited to acrylonitrile butadiene rubber (NBR), natural rubber (NR) and polychloroprene (CR));
a dispersing agent (such as but not limited to disodium N-octadecylsulphosuccinamate, salt of naphthalene sulphonic acid, and sodium silicaflouride aqueous dispersion);
a stabilizer (such as but not limited to sodium lauryl sulphate and monosodium salt of sulphated methyl oleate);
a pH adjuster (such as but not limited to ammonia, sodium hydroxide, potassium hydroxide);
an accelerator (such as but not limited to zinc dibutyldithiocarbamate and zinc diethyldithiocarbamate);
a crosslinking agent (such as but not limited to sulphur (primary) and/or zinc oxide (secondary));
a pigment (such as but not limited to titanium dioxide);
a filler, reinforcing agent (such as but not limited to silicate-based filler or calcium carbonate);
an antioxidant (such as but not limited to polymeric phenolic);
a mould release agent (such as but not limited to blend of paraffin wax-emulsion;
an antifoaming agent (such as but not limited to methylcyclotetrasiloxane and/or dimethyl siloxane); and
a wetting agent (such as but not limited to t-octylphenoxypolyethoxyethanol and/or polyethylene glycol sorbitan monostearate).

Tables 2, 4 to 6 show chemical components (as listed above) and compositions thereof used in preparing the first and the second latex formulations.

TABLE 2
Chemical components and compositions thereof to prepare
the first and the second latex formulations of NBR

| Chemical | Working range (*phr) |
|---|---|
| NBR | 100.00 |
| Dispersing agent | 0.60 to 1.40 |
| pH adjuster | 1.80 to 2.60 |
| Primary crosslinking agent | 2.00 to 4.00 |
| Secondary crosslinking agent | 0.30 to 1.00 |
| Accelerator | 0.60 to 1.50 |
| Pigment | 0.60 to 1.50 |
| Antifoaming agent and wetting agent | 0.02 to 0.07 |
| Filler/reinforcing agent | 2.00 to 8.00 |

*parts per hundred rubber

TABLE 3
Chemical components and compositions thereof to prepare
the coagulant layer for NBR latex formulations

| Chemical | Working range (Weight %) |
|---|---|
| Wetting agent | 0.050 to 0.250 |
| Former release agent | 1.000 to 3.000 |
| Latex coagulating agent | 8.000 to 12.000 |
| pH adjuster | 0.025 to 0.050 |
| Diluent | 84.700 to 90.925 |

TABLE 4
Chemical components and compositions thereof to prepare
the first and the second latex formulations of NR

| Chemical | Working range (*phr) |
|---|---|
| NR | 100.00 |
| pH adjuster | 0.05 to 0.40 |
| Filler/reinforcing agent | 35.00 to 55.00 |
| Accelerator | 0.10 to 0.30 |
| Primary crosslinking agent | 1.00 to 2.00 |
| Secondary crosslinking agent | 0.70 to 1.50 |
| Antifoaming agent | 0.03 to 0.20 |
| Mould release agent | 0.60 to 1.50 |
| Dispersing agent | 0.10 to 0.30 |

*parts per hundred rubber

TABLE 5
Chemical components and compositions thereof to prepare
the coagulant layer for NR latex formulations

| Chemical | Working range (Weight %) |
|---|---|
| Former release agent | 3.800 to 5.000 |
| Latex coagulating agent | 3.000 to 8.000 |
| Wetting agent | 0.050 to 0.250 |
| pH adjuster | 0.025 to 0.050 |
| Diluent | 86.700 to 93.125 |

TABLE 6
Chemical components and compositions thereof to prepare the first and
the second latex formulations of CR

| Chemical | Working range (*phr) |
|---|---|
| CR | 100.00 |
| Wetting agent | 0.05 to 0.15 |
| Stabilizer | 0.30 to 0.70 |
| Dispersing agent | 6.00 to 10.00 |
| Crosslinking agent | 0.70 to 1.50 |
| Antioxidant | 0.20 to 0.70 |
| Pigment | 0.30 to 0.80 |

*parts per hundred rubber

TABLE 7
Chemical components and compositions thereof to prepare
the first coagulant layer for CR latex formulations

| Chemical | Working range (Weight %) |
|---|---|
| Former release agent | 3.000 to 4.000 |
| Wetting agent | 0.004 to 0.008 |
| Latex coagulating agent | 10.000 to 18.000 |
| pH adjuster | 0.025 to 0.050 |
| Diluent | 77.942 to 86.971 |

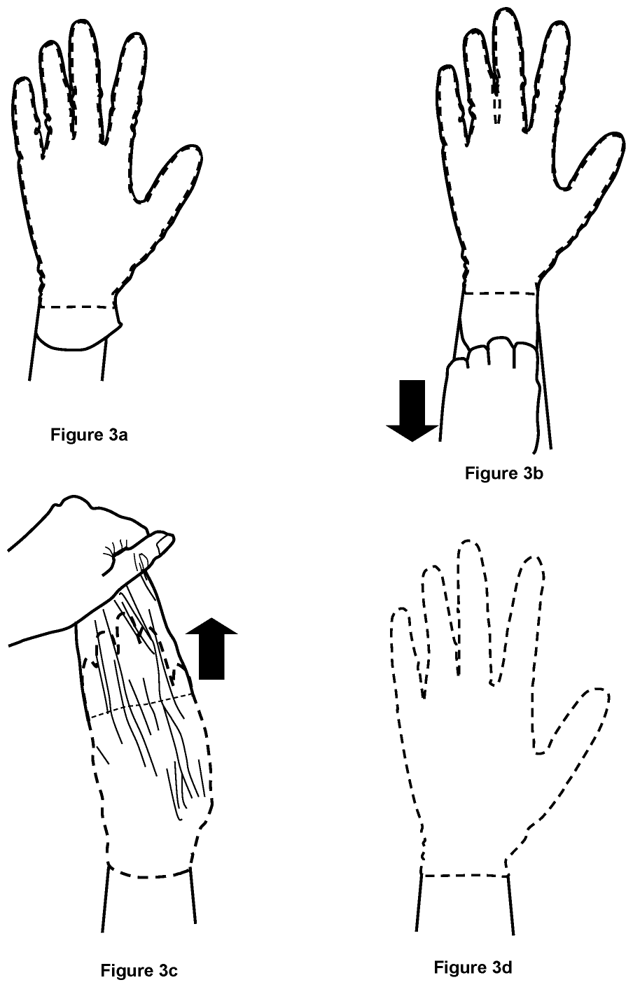

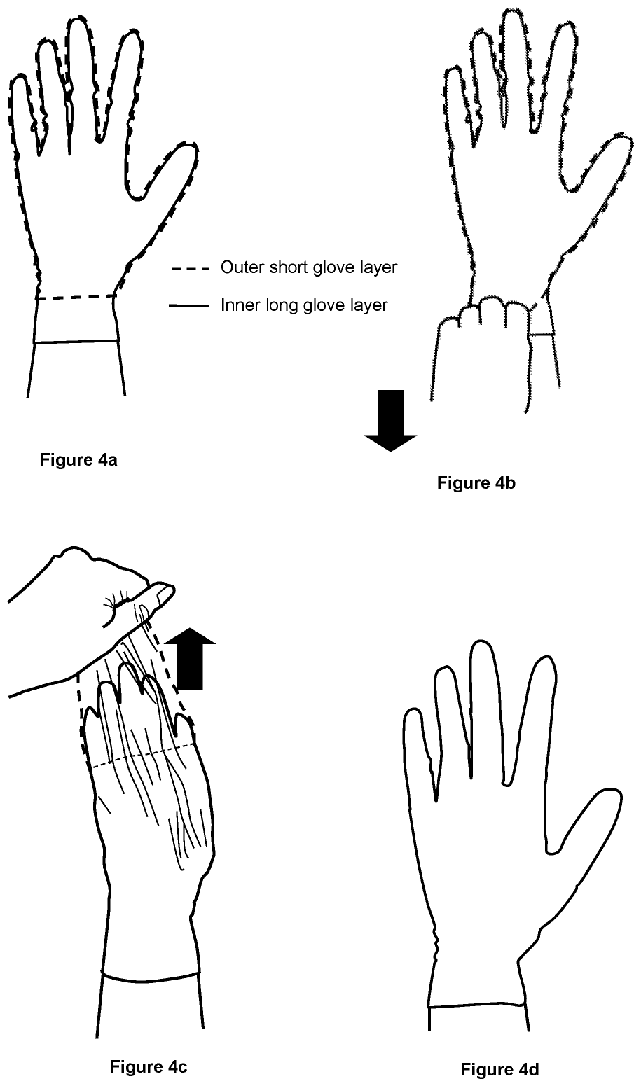

A third embodiment of the present invention relates to a method of peeling an outer glove layer of a detachable bi-layered glove from an inner glove layer of the detachable bi-layered glove. Referring to the accompanying drawings, FIGS. 3a to 3d as well as FIGS. 4a to 4d are illustrative figures showing the method of peeling off the outer glove layer of the detachable bi-layered glove from the inner glove layer of the detachable bi-layered glove, wherein the method comprises the steps of:
i. pulling cuff area of the outer glove layer until its full circumference is detached from the inner glove layer and
ii. peeling off the outer glove layer once the full circumference of the outer glove layer is detached from the inner glove layer to yield the inner glove layer for further usage.

The following example is constructed to illustrate an embodiment of the present invention in a non-limiting sense.

A detachable bi-layered glove of the present invention comprises an outer glove layer and an inner glove layer that is separated by an adhesive layer, wherein the gloves are prepared using latex formulations as described in Tables 2, 4 and 6 adopting a method commonly known in the glove manufacturing industry, wherein the adhesive layer is as described in Table 1 and wherein the outer and the inner gloves are in different lengths, wherein the longer glove has a standard length ranging between 270 mm to 290 mm and wherein the shorter glove has a standard length ranging between 230 mm to 250 mm.

### Test Results for all the Gloves

Table 8 shows the survey results from 20 respondents on the peeling efficiency of the detachable bi-layered glove of the present invention.

TABLE 8
Peeling efficiency of the detachable bi-
layered glove of the present invention

| Detachable glove latex type | Peeling efficiency |
|---|---|
| Acrylonitrile butadiene rubber (NBR) | Very good |
| Polychloroprene rubber (CR) | Very good |
| Natural rubber (NR) | Good |

Based on user's experience, it is evident that the users are finding the peeling efficiencies are very good for both the NBR and the CR as compared to NR detachable bi-layered gloves.

Tables 9 and 10 shows the survey results obtained from 20 respondents on peeling efficiencies of detachable bi-layered glove of the present invention when the inner layer and outer layer are in different lengths with different adhesive layers are applied between the detachable bi-layered glove of the present invention. The peeling efficiencies are rated from 1 to 3, wherein 1 signifies easy peeling, 2 signifies normal peeling and 3 signifies hard peeling.

TABLE 9
Peeling efficiencies of the detachable bi-layered glove of the
present invention when different adhesive layers are applied
between the detachable bi-layered glove of the present invention
with a longer outer layer and a shorter inner layer

| | | | Tearing during peeling | |
|---|---|---|---|---|
| | Peeling efficiency | | of outer layer | |
| | Adhesive | | Adhesive | |
| | layer of | Conventional | layer of | Conventional |
| Detachable glove | the present | adhesive | the present | adhesive |
| latex type | invention | layer | invention | layer |
| Acrylonitrile | 1 | 3 | No | Yes |
| butadiene rubber | | | | |
| (NBR) | | | | |
| Polychloroprene | 1 | 3 | No | Yes |
| rubber (CR) | | | | |
| Natural rubber | 2 | 3 | No | Yes |
| (NR) | | | | |

TABLE 10
Peeling efficiencies of the detachable bi-layered glove of the
present invention when different adhesive layers are applied
between the detachable bi- layered glove of the present invention
with a shorter outer layer and a longer inner layer

| | | | Tearing during peeling | |
|---|---|---|---|---|
| | Peeling efficiency | | of outer layer | |
| | Adhesive | | Adhesive | |
| | layer of | Conventional | layer of | Conventional |
| Detachable glove | the present | adhesive | the present | adhesive |
| latex type | invention | layer | invention | layer |
| Acrylonitrile | 1 | 3 | No | Yes |
| butadiene rubber | | | | |
| (NBR) | | | | |
| Polychloroprene | 1 | 3 | No | Yes |
| rubber (CR) | | | | |
| Natural rubber | 2 | 3 | No | Yes |
| (NR) | | | | |

Based on users' experience, it is evident that the users are finding the peeling efficiencies are good for all the detachable bi-layered gloves with an adhesive layer of the present invention applied between the detachable bi-layered gloves.

As a whole, the detachable bi-layered glove of the present invention is able to overcome the conventional shortcomings since the glove of the present invention is able to cater users to conveniently use the same piece of glove twice without the risk of contamination and without the hassle of doffing and donning twice.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises”, “comprising”, “including”, and “having” are inclusive and therefore specify the presence of stated features, integers, steps, operations, elements, and/or components, but do not preclude the presence or addition of one or more other features, integers, steps, operations, elements, components, and/or groups thereof.

The method steps, processes, and operations described herein are not to be construed as necessarily requiring their performance in the particular order discussed or illustrated, unless specifically identified as an order of performance. It is also to be understood that additional or alternative steps may be employed. The use of the expression “at least” or “at least one” suggests the use of one or more elements, as the use may be in one of the embodiments to achieve one or more of the desired objects or results.

What is claimed is:

1. A detachable bi-layered glove is comprising an outer glove layer and an inner glove layer that is separated by an adhesive layer, wherein the adhesive layer includes a wetting agent, a latex coagulating agent, a synthetic organic polymer and water, wherein the wetting agent is used in an amount ranging between 0.40% to 0.80% by weight of the adhesive layer, wherein the latex coagulating agent is used in an amount ranging between 17.00% to 20.00% by weight of the adhesive layer, wherein the synthetic organic polymer is used in an amount ranging between 10.00% to 14.00% by weight of the adhesive layer and wherein water is used in an amount ranging between 65.20% to 72.60% by weight of the adhesive layer.

2. The detachable bi-layered glove as claimed in claim 1, wherein the wetting agent is used in an amount ranging between 0.60% to 0.70% by weight of the adhesive layer.

3. The detachable bi-layered glove as claimed in claim 1, wherein the wetting agent is selected from the group consisting of non-ionic surfactant, blends of non-ionic surfactant and mixtures thereof.

4. The detachable bi-layered glove as claimed in claim 3, wherein the non-ionic surfactant is selected from the group consisting of fatty acid ethoxylate, lauric acid ethoxylate, oleic acid ethoxylate, other similar compounds and mixtures thereof.

5. The detachable bi-layered glove as claimed in claim 3, wherein the blends of non-ionic surfactant are polysiloxane surfactant which is selected from the group consisting of methyl (propylhydroxide ethoxylated) bis (trimethylsiloxy) silane, other similar compounds and mixtures thereof.

6. The detachable bi-layered glove as claimed in claim 1, wherein the latex coagulating agent is used in an amount ranging between 17.50% to 19.50% by weight of the adhesive layer.

7. The detachable bi-layered glove as claimed in claim 1, wherein the latex coagulating agent is selected from the group consisting of calcium nitrate, zinc nitrate, calcium chloride and mixtures thereof.

8. The detachable bi-layered glove as claimed in claim 1, wherein the synthetic organic polymer is used in an amount ranging between 12.00% to 13.00% by weight of the adhesive layer.

9. The detachable bi-layered glove as claimed in claim 1, wherein the synthetic organic polymer is selected from the group consisting of styrene acrylic copolymer emulsion, polymethyl methacrylate emulsion, low-density polyethylene emulsion, high-density polyethylene emulsion, polypropylene emulsion, polystyrene emulsion, polyurethane emulsion and mixtures thereof.

10. The detachable bi-layered glove as claimed in claim 1, wherein water is used in an amount ranging between 66.80% to 69.90% by weight of the adhesive layer.

11. A detachable multi-layered glove, wherein the multi-layered glove is of at least 3 layers, wherein the layers are separated by an adhesive layer, wherein the adhesive layer includes a wetting agent, a latex coagulating agent, a synthetic organic polymer and water, wherein the wetting agent is used in an amount ranging between 0.40% to 0.80% by weight of the adhesive layer, wherein the latex coagulating agent is used in an amount ranging between 17.00% to 20.00% by weight of the adhesive layer, wherein the synthetic organic polymer is used in an amount ranging between 10.00% to 14.00% by weight of the adhesive layer and wherein water is used in an amount ranging between 65.20% to 72.60% by weight of the adhesive layer.

12. A detachable bi-layered glove as claimed in claim 1, wherein the detachable bi-layered glove is a chlorinated glove.

13. A detachable bi-layered glove as claimed in claim 10, wherein the detachable bi-layered glove is a chlorinated glove.

14. A method of manufacturing a chlorinated detachable bi-layered glove, wherein the method comprises the steps of:
i. dipping a former into a coagulant solution to produce a coagulant layer coated on the former;
ii. drying the coagulant layer coated on the former obtained in step (i) to produce a dried coagulant layer coated on the former;
iii. dipping the dried coagulant layer coated on the former obtained in step (ii) into first latex formulation to produce a first latex layer coated on the former;
iv. drying the first latex layer coated on the former obtained in step (iii) to produce a dried first latex film coated on the former;
v. beading the dried first latex film coated on the former obtained in step (iv) to produce a beaded latex film coated on the former;
vi. curing the beaded latex film coated on the former obtained in step (v) to produce a cured latex film coated on the former;
vii. dipping the cured latex film coated on the former obtained in step (vi) into an adhesive solution to produce an adhesive layer coated on the first latex film;
viii. drying the adhesive layer coated on the first latex film obtained in step (vii) to produce a dried adhesive layer coated on the first latex film;
ix. dipping the dried adhesive layer coated on the first latex film obtained in step (viii) into second latex formulation to produce a second latex layer coated on the adhesive layer;
x. drying the second latex layer coated on the adhesive layer obtained in step (ix) to produce a dried second latex film coated on the adhesive layer;
xi. pre-leaching the dried second latex film coated on the adhesive layer obtained in step (x) to produce a pre-leached detachable bi-layered latex film coated on the former;
xii. beading the pre-leached detachable bi-layered latex film coated on the former obtained in step (xi) to produce a beaded detachable bi-layered latex film coated on the former;
xiii. curing the beaded detachable bi-layered latex film coated on the former obtained in step (xiii) to produce a cured detachable bi-layered glove coated on the former;
xiv. cooling the cured detachable bi-layered glove coated on the former obtained in step (xiii) to produce a cooled detachable bi-layered glove coated on the former;
xv. chlorinating the cooled detachable bi-layered glove coated on the former obtained in step (xiv) to produce a chlorine treated detachable bi-layered glove coated on the former;
xvi. post-leaching the chlorine treated detachable bi-layered glove coated on the former obtained in step (xv) to produce a post-leached detachable bi-layered glove coated on the former; and
xvii. drying the post-leached detachable bi-layered glove coated on the former obtained in step (xvi) to produce the chlorinated detachable bi-layered glove,
wherein the adhesive layer produced in the step (vii) includes a wetting agent, a latex coagulating agent, a synthetic organic polymer and water, wherein the wetting agent is used in an amount ranging between 0.40% to 0.80% by weight of the adhesive layer, wherein the latex coagulating agent is used in an amount ranging between 17.00% to 20.00% by weight of the adhesive layer, wherein the synthetic organic polymer is used in an amount ranging between 10.00% to 14.00% by weight of the adhesive layer and wherein water is used in an amount ranging between 65.20% to 72.60% by weight of the adhesive layer.

15. A detachable bi-layered glove as claimed in claim 1, wherein the detachable bi-layered glove is a polymer coated glove.

16. A detachable bi-layered glove as claimed in claim 10, wherein the detachable bi-layered glove is a polymer coated glove.

17. A method of manufacturing a polymer coated detachable bi-layered glove, wherein the method comprises the steps of:
i. dipping a former into a coagulant solution to produce a coagulant layer coated on the former;
ii. drying the coagulant layer coated on the former obtained in step (i) to produce a dried coagulant layer coated on the former;
iii. dipping the dried coagulant layer coated on the former obtained in step (ii) into first latex formulation to produce a first latex layer coated on the former;
iv. drying the first latex layer coated on the former obtained in step (iii) to produce a dried first latex film coated on the former;
v. beading the dried first latex film coated on the former obtained in step (iv) to produce a beaded latex film coated on the former;
vi. curing the beaded latex film coated on the former obtained in step (v) to produce a cured latex film coated on the former;
vii. dipping the cured latex film coated on the former obtained in step (vi) into an adhesive solution to produce an adhesive layer coated on the first latex film;
viii. drying the adhesive layer coated on the first latex film obtained in step (vii) to produce a dried adhesive layer coated on the first latex film;
ix. dipping the dried adhesive layer coated on the first latex film obtained in step (viii) into second latex formulation to produce a second latex layer coated on the adhesive layer;
x. drying the second latex layer coated on the adhesive layer obtained in step (ix) to produce a dried second latex film coated on the adhesive layer;
xi. pre-leaching the dried second latex film coated on the adhesive layer obtained in step (x) to produce a pre-leached detachable bi-layered latex film coated on the former;
xii. dipping the pre-leached detachable bi-layered latex film obtained in step (xi) into polymer solution to produce a polymer coated detachable bi-layered latex film;
xiii. beading the polymer coated detachable bi-layered latex film coated on the former obtained in step (xii) to produce a beaded detachable bi-layered latex film coated on the former;
xiv. curing the beaded detachable bi-layered latex film coated on the former obtained in step (xiii) to produce a cured detachable bi-layered glove coated on the former;
xv. post-leaching the cured detachable bi-layered glove coated on the former obtained in step (xiv) to produce a post-leached detachable bi-layered glove coated on the former;
xvi. drying the post-leached detachable bi-layered glove coated on the former obtained in step (xvi) to produce the polymer coated detachable bi-layered glove,
wherein the adhesive layer produced in the step (vii) includes a wetting agent, a latex coagulating agent, a synthetic organic polymer and water, wherein the wetting agent is used in an amount ranging between 0.40% to 0.80% by weight of the adhesive layer, wherein the latex coagulating agent is used in an amount ranging between 17.00% to 20.00% by weight of the adhesive layer, wherein the synthetic organic polymer is used in an amount ranging between 10.00% to 14.00% by weight of the adhesive layer and wherein water is used in an amount ranging between 65.20% to 72.60% by weight of the adhesive layer.

18. A method of peeling the detachable bi-layered glove of claim 1, wherein the method comprises the steps of:
i. pulling cuff area of the outer glove layer until its full circumference is detached from the inner glove layer; and
ii. peeling off the outer glove layer once the full circumference of the outer glove layer is detached from the inner glove layer to yield the inner glove layer for further usage.

19. A method of peeling the detachable bi-layered glove of claim 10, wherein the method comprises the steps of:
iii. pulling cuff area of the outer glove layer until its full circumference is detached from the inner glove layer; and
iv. peeling off the outer glove layer once the full circumference of the outer glove layer is detached from the inner glove layer to yield the inner glove layer for further usage.
